(19) United States
(12) Patent Application Publication
(10) Pub. No.: US 2017/0000717 A1
(43) Pub. Date: Jan. 5, 2017

(54) A COMPOSITION OF MATTER AND METHOD OF USE FOR A NUTRITIONAL SUPPLEMENT

(71) Applicants: Esther Reynoso, (US); Fabio R. Magnelli, (US)

(72) Inventor: Esther Reynoso, El Cajon, CA (US)

(73) Assignees: Esther Reynoso, El Cajon, CA (US); Fabio R. Magnelli, El Cajon, CA (US)

(21) Appl. No.: 15/197,241

(22) Filed: Jun. 29, 2016

Related U.S. Application Data
(60) Provisional application No. 62/186,714, filed on Jun. 30, 2015.

(51) Int. Cl. A61K 8/67; A61K 8/23; A61Q 19/08; A61K 8/24; A61Q 19/00; A61K 8/36; A61K 8/19

(52) U.S. Cl. CPC A61K 8/673; A61K 8/67; A61K 8/671; A61K 8/676; A61K 8/361; A61K 8/23; A61K 8/19; A61K 8/24; A61Q 19/00; A61Q 19/08; A61K 2800/59

(57) ABSTRACT

Disclosed is a composition of matter and method of use for a nutritional supplement having at least thee components. A first component being one or more vitamins, wherein at least one of the one or more vitamins is vitamin B, wherein at least one of the one or more vitamins is vitamin D, and wherein at least one of the one or more vitamins is vitamin A. A second component comprising one or more acids, and a third component comprising one or more oils, wherein the first component, the second component, and the third component are combined in a homogenous mixture.

### CROSS-REFERENCE TO RELATED APPLICATION(S)

The present application claims priority to U.S. Provisional Patent Application No. 62/186,714 filed on Jun. 30, 2015, entitled NUTRITIONAL SUPPLEMENT FOR SKIN, the entire disclosure of which is incorporated by reference herein.

### BACKGROUND OF THE INVENTION

1. Field of Invention

This invention relates to nutrition for the skin, particular vitamins and minerals that are applied to the skin

2. Description of Related Art

Aging and the passing of time have effects on all body systems. While each system serves a vital role in human life, the skin is arguably the most crucial system. It is generally understood that skin is the largest organ in the human body and provides a barrier to physical injury and pathogens. Overtime, the composition of the skin is subject to change and lose efficacy. The anatomy of the skin generally includes the epidermis, dermis, and hypodermis as well as an abundant source of pluripotent stem cells.

Each of these layers comprises a number of components such as the melanocytes and keratinocytes of the epidermis, which is the avascular outermost layer. Elastin and collagen can be found in the dermis, while the hypodermis contains fat cells and abuts muscular sub layers. Sebaceous glands and hair follicles extend generally between and through the dermis and epidermis.

With age, major deconstruction of the elastin and collagen in the dermis leads to irregularities in the epidermis causing a breakdown in the efficacy and aesthetics of the skin overall. This breakdown results in thinning of the layers along with a decreased ability to repair and absorb necessary elements

While there is significant prior art in the field of skin car, particularly skin crémes or lotions that provide vitamins and minerals that are necessary for the skin. Aging depletes skin of the elasticity and resilience that it enjoys in a more youthful state, and various lotions and crémes, as well as other treatments (masks, etc.). However, the prior art has been unable to rejuvenate skin to restore elasticity, shine and resilience. Some topical applications merely seek to cover the physical signs of epidermal breakdown by smoothing deformations in the exterior integrity or covering up discoloration.

Based on the foregoing, there is a need in the art for a reparative solution to actively effect the physical changes to the dermal layers while preventing unnecessary side effects resulting from harmful chemical treatments.

### SUMMARY OF THE INVENTION

A composition of matter for a nutritional supplement comprising a first component comprising one or more vitamins, wherein at least one of the one or more vitamins is vitamin B, wherein at least one of the one or more vitamins is vitamin D, and wherein at least one of the one or more vitamins is vitamin A; a second component comprising one or more acids; and a third component comprising one or more oils, wherein the first component, the second component, and the third component are combined in a homogenous mixture.

In an embodiment, the one or more vitamins are selected from the group consisting of: vitamin A, vitamin B, vitamin C, vitamin D, and vitamin E, wherein vitamin B is a complex comprising vitamin B3, vitamin B6, vitamin B9, and vitamin B12.

In an embodiment, the one or more acids are selected from the group consisting of palmitic acid, stearic acid, and linoleic acid.

In an embodiment, the one or more oils are argan oil or fish oils.

In an embodiment, the composition further comprises one or more antioxidants, wherein the one or more antioxidants are tocopherols or tocotrienols.

In an embodiment, the composition further comprises at least one component selected from the group consisting of beta amyne, butyrospermum, lupeol, iron, zinc and magnesium.

In an embodiment, a method of using a composition of matter for a nutritional supplement comprising the steps of: a user combining three or more components, wherein the three or more components comprise at least one vitamin, at least one acid, and at least one oil; forming a homogenous mixture with the three or more components; applying the homogenous mixture to the user's skin, wherein the user selects a body region for treatment; massaging the homogenous mixture onto the skin; the homogenous mixture engaging the user's skin, wherein one or more of the three or more components contact at least one layer of the user's skin; and the user waiting a period of time sufficient for the homogenous mixture to be absorbed.

In an embodiment, the method further comprises the steps of: the user separating one or more portions of a sheet of material for mask application, wherein the sheet of material comprises the one or more potions, and wherein the an outer perimeter of the one or more portions are perforated; the user saturating the one or more portions with the homogenous mixture; and the user applying the saturated one or more portions to the user's face.

In an embodiment, the homogenous mixture comprises: Omega 3; Phosphorous; Selenium; Vitamin B6; Vitamin D; Vitamin B9; Calcium; Iron; Zinc; Omega 3 DHA; Omega 3 EPA; Vitamin A; and Vitamin B12.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a view of the top plan view of the nutritional supplement mask, according to an embodiment of the present invention.

### DETAILED DESCRIPTION OF PREFERRED EMBODIMENTS

A nutritional supplement for the skin is a homogenous mixture of a plurality of different agents having various effects based on the chemical interaction with surrounding cells and extracellular environments. The combination of the different agents results in extraordinary effects such as increased hydration, elasticity, and resilience.

In an embodiment, the different agents include combinations of vitamins: A, B, C, and D with minerals such as: phorphous, selenium, calicium, iron, and zinc. In another embodiment, various fatty acids are incorporated into the mixture. Examples of these fatty acids are omega fatty acids derived from various fish species.

In another embodiment additives are incorporated into the mixture. Examples of these additives are

In an embodiment various application techniques are used based on the location of the body to be treated. For example, a mask is prepared for application to a user's face. A lotion or créme is prepared for manual application to various other regions of the body.

In preparation of a mask, for facial treatment, the nutritional supplement is prepared by combining the various components into a mixture where liquid components of various viscosities are combined and any solid component is introduced to the liquid mixture. In an embodiment, solid components dissolve into the liquid mixture. In an alternative embodiment, solid components do not dissolve into the mixture and remain in their solid state for exfoliation or topical activity.

Once the mixture is prepared, incorporating all components, the user applies the nutritional supplement to an absorbent material. For example, the absorbent material may be a paper product, cloth or other textile, or other porous material appreciated by one skilled in the art having the capability of taking up the nutritional supplement. Once the supplement has been combined with the material, the user applies the impregnated material to the user's face. In this way, the material is saturated with the nutritional supplement and when contact is made between the material and the user's face, transfer from the saturated material to the user's face occurs. The user waits a period of time for the nutritional supplement to dehydrate or transform from a liquid state to a more static and solid state. At this point, the nutritional supplement is effectively indicating that it is time for the mask to be removed. This reaction is a result of combination of the supplement with the epidermis and the transport of various components of the mixture across the layers of the skin through either active or passive transport. Active transport being the particular component's ability to engage the cellular structure of the dermal layers.

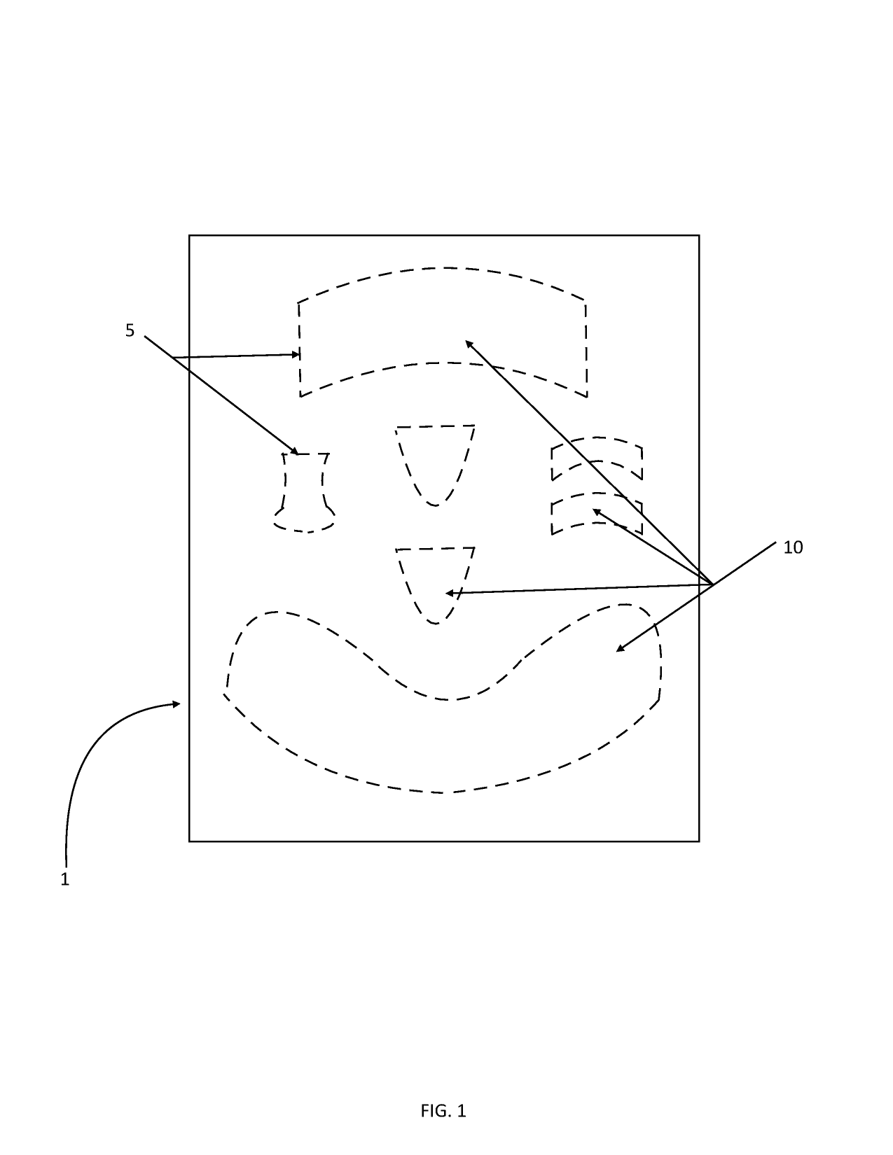

In an embodiment, illustrated in FIG. 1, the absorbent material is cut by the user for a particular application to appreciate the unique contours of the user's face. In another embodiment, the absorbent material is a single sheet 1 of material with a plurality of perforations 5 outlining various shapes 10 for an application specific size. In this embodiment, the user grasps the absorbent material and selects the particular shape to contour the region of their face to receive the treatment. The user applies pressure to area the sheet to be removed. The pressure applied is sufficient to overcome the connection between the region to be removed and the remaining portion of the sheet of material. A user can either saturate the specific removed portion of the sheet of material with the nutritional supplement, or they can saturate the entire sheet of material prior to removing an contoured portions.

In another embodiment, the lotion is manually applied to a user's skin. The nutritional supplement is prepared in a similar fashion to preparing the moisture for the mask application. However, rather than apply the material to an intermediary material, a user acquires a desired amount for a particular body region coverage and applies the amount of nutritional supplement directly to the skin.

In an embodiment, the amount of nutritional supplement applied to the skin transforms from a liquid to a solid state. Once the material has converted in this manner, the user is free to remove the nutritional supplement.

In an embodiment, the nutritional supplement using a single treatment of the mask, incorporates the following components added to a mask base, or absorbent material:

Vitamin A in the amount of 490 IU
Vitamin D in the amount of 54 IU

### Folate/Folic Acid

### Choline or Vitamin B9

Selenium in the amount of 18 miligrams (mg)
Potassium in the amount of 66 mg
Phosphorous in the amount of 36 mg
Calcium in the amount of 44 mg
Iron in the amount of 2 mg
Magnesium in the amount of 2 mg
Zinc in the amount of 2 mg
Copper in the amount of 2 mg
Shark, pink salmon and fish oils are added in the amount of 240 drops. These oils provide anti-oxidants which permits cell repair and further resilience for the skin to which it is applied.

In another embodiment, the nutritional supplement using a single treatment of the mask, incorporates the following components added to a mask base, or absorbent material:

Omega 3 in the amount of 6000 mg
Phosphorous in the amount of 18 mg
Selenium in the amount of 18 mg
Vitamin B6 in the amount of 27 mg
Vitamin D in the amount of 20 mg
Vitamin B9 in the amount of 20 mg
Calcium in the amount of 43 mg
Iron in the amount of 18 mg
Zinc in the amount of 18 mg
Omega 3 DHA (eicosa pentanoic acid) in the amount of 2000 mg
Omega 3 EPA (docosahexaenoic acid) in the amount of 1900 mg
Vitamin A in the amount of 502 mg
Vitamin B12 in the amount of 36 mg

In another embodiment, the nutritional supplement is made per use by combining:

Omega 3 in the amount of 200+/−1-10 drops, wherein the drops are taken from a standard dropper
Phosphorus in the amount of 18% of the total mixture volume
Selenium in the amount of 18% of the total mixture volume
Vitamin B Complex (i.e. 6, 9, 12) in the amount of 28% of the total mixture volume
Vitamin A in the amount of 120+/−1-10 drops, wherein the drops are taken from a standard dropper
Vitamin D3 in the amount of 120+/−1-10 drops, wherein the drops are taken from a standard dropper
Iron in the amount of 18% of the total mixture volume
Calcium in the amount of 16% of the total mixture volume
Zinc in the amount of 0.5% of the total mixture volume.

In an embodiment, the percentages of the included components has a variation of +/−3 percent.

In an alternative embodiment, a lotion or créme is prepared for daily use where the following components are combined and mixed thoroughly forming a homogenous mixture:

Vitamin B complex (i.e. B3, B6, B9, B12) in the amount of 15 mL
A proprietary oil complex totaling an amount of 53 mL composed of:

Moroccan Aragan Oil with or without oleic 42.8%

Linoleic 36.8%

Palmitic acid 12.0%

Stearic acid 60%

Linoleic acid making up 0.05%

Vitamin E in the amount of 38 mL
Vitamin D3 in the amount of 8.5 mL
Vitamin A in the amount of 8.5 mL

In another embodiment, the lotion is also composed of equal parts of the Vitamin B complex, 1.5 mG of Vitamin D, 20 mg of Vitamin A, 20 mg of Vitamin C, 50 mg of Argan oil, 50 mg of Vitamin E, Antioxidant tocopherols (beta amyne, butyrospermum, lupeol, magnesium), 6 mg of Vitamin B1, Vitamin D epa (cicosa pentanoic acid), and 15 mg of lineoleic acid.

The second step is a créme or lotion that is massaged into the skin that was first masked. The following ingredients are combined:

Vitamins B9 and B12 in the amount of 1 fluid ounce
Vitamin D in the amount of 1 fluid ounce
Pure virgin of Vitamin E in the amount of 1 fluid ounce
Moroccan argan oil in the amount of 1 fluid ounce, and

### Keratin

In an embodiment, the lotion or cream is applied to the skin multiple times per day, wherein the user grabs an amount sufficient to cover a particular body region. Then the user applies the amount to the body region and massages the lotion onto the surface of the skin. The lotion then penetrates the skin through pores, via active or passive uptake where each layer of the skin is effected by the various components.

In various embodiments, the regions of the skin where the nutritional supplement is applied include a user's face, lips, neck, chest, arm, underarms, hands, legs, feet, and back. When applied, the lotion is massages for an amount of time until the nutritional supplement is absorbed and no longer has a shiny appearance.

The créme that is formed (Food for the Skin) fights gravity of the skin by tightening up the elasticity of the skin and shrinking the pores. It rejuvenates and nurtures the skin through interacting with the dermal layers. For example, the elastin and collagen which support elastic properties of the skin breakdown with age and this nutritional supplement adds essential vitamins, minerals, and nutrients in a unique combination for direct application to various areas. The unique combination allows for a similar benefit through the different regions of the body regardless of the thickness, density, or composition of the particular skin of the region. A specific example of one of the components is Choline's effect in the repair of DNA.

In order to use the product, layers of the mask are applied to the face, the mouth, neck, chest, ears, arms, shoulders and hands, anywhere the skin is dry and wrinkled in appearance. The mask is covered with small squares or mask paper, which facilitate removal of the mask. The mask is applied to these parts and left for an hour, during which time the nutrients are absorbed into the skin. The mask is then removed, and the remaining particles of the mask are removed using a small face towel. Fruit juice having astringent properties such as grapefruit juice is then used to wash the skin. The skin parts that were masked, are then massaged with the lotion of step two. The lotion is left until the skin absorbs it, and the procedure may be followed up to three times daily in order to provide the greatest benefit. Often people over 40 years old begin to develop dry skin and may benefit from such treatment.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A composition of matter for a nutritional supplement comprising:
a. a first component comprising one or more vitamins, wherein at least one of the one or more vitamins is vitamin B, wherein at least one of the one or more vitamins is vitamin D, and wherein at least one of the one or more vitamins is vitamin A;
b. a second component comprising one or more acids; and
c. a third component comprising one or more oils,
wherein the first component, the second component, and the third component are combined in a homogenous mixture.

2. The compositions of claim 1, wherein the one or more vitamins are selected from the group consisting of: vitamin A, vitamin B, vitamin C, vitamin D, and vitamin E, wherein vitamin B is a complex comprising vitamin B3, vitamin B6, vitamin B9, and vitamin B12.

3. The compositions of claim 1, wherein the one or more acids are selected from the group consisting of palmitic acid, stearic acid, and linoleic acid.

4. The compositions of claim 1, wherein the one or more oils are argan oil or fish oils.

5. The compositions of claim 2, wherein the one or more acids are selected from the group consisting of palmitic acid, stearic acid, and linoleic acid.

6. The compositions of claim 5, wherein the one or more oils are argan oil or fish oils The compositions of claim 1, further comprising one or more antioxidants, wherein the one or more antioxidants are tocopherols or tocotrienols.

7. The composition of claim 7, further comprising at least one component selected from the group consisting of beta amyne, butyrospermum, lupeol, iron, zinc and magnesium.

8. A method of using a composition of matter for a nutritional supplement comprising the steps of:
a. a user combining three or more components, wherein the three or more components comprise at least one vitamin, at least one acid, and at least one oil;
b. forming a homogenous mixture with the three or more components;
c. applying the homogenous mixture to the user's skin, wherein the user selects a body region for treatment;
d. massaging the homogenous mixture onto the skin;
e. the homogenous mixture engaging the user's skin, wherein one or more of the three or more components contact at least one layer of the user's skin; and
f. the user waiting a period of time sufficient for the homogenous mixture to be absorbed.

9. The method of claim 9, further comprising the steps of:
a. the user separating one or more portions of a sheet of material for mask application, wherein the sheet of material comprises the one or more potions, and wherein the an outer perimeter of the one or more portions are perforated;
b. the user saturating the one or more portions with the homogenous mixture; and
c. the user applying the saturated one or more portions to the user's face.

10. The method of claim 10, wherein the homogenous mixture further comprises:
a. Omega 3;
b. Phosphorous;
c. Selenium;
d. Vitamin B6;
e. Vitamin D;
f. Vitamin B9;
g. Calcium;
h. Iron;
i. Zinc;
j. Omega 3 DHA;
k. Omega 3 EPA;
l. Vitamin A; and
m. Vitamin B12.
